(19) United States
(12) Patent Application Publication
(10) Pub. No.: US 2005/0027172 A1
(43) Pub. Date: Feb. 3, 2005

(54) Method and computer program for conducting comprehensive assessment of dental treatment and generating diagnostic opinion

(76) Inventors: Mark Benavides, Ottawa, IL (US); John C. Kois, Federal Way, WA (US)

Correspondence Address: JOSEPH S. HEINO, ESQ.;DAVIS & KUELTHAU, S.C., 111 E. KILBOURN, SUITE 1400, MILWAUKEE, WI, 53202-6613, US

(21) Appl. No.: 10/628,186

(22) Filed: Jul. 28, 2003

(51) Int. Cl. A61B 5/00; G06F 17/60

(52) U.S. Cl. 600/300; 705/2; 128/920

(57) ABSTRACT

A computer implemented method and system is provided whereby, in the preferred embodiment, dental data is input via a series of questions and prompts through a custom program written in a high level language. The questions answered and the data gathered is stored in a relational database, and every answer is given a numerical value. The values are eventually output to a spreadsheet where various math functions and custom algorithms are used to populate several worksheets. The output worksheets generated contain one or more diagnostic opinions that include specific and generalized prognoses and risk assessment for the patient's dental conditions.

### FIELD OF THE INVENTION

The present invention relates generally to the field of computer implemented methods and systems for processing medical information. More particularly, it relates to a computer implemented method and program for comprehensively assessing the health of a dental patient and then generating a diagnostic opinion for treatment of that patient.

### BACKGROUND OF THE INVENTION

Dental treatment, and medical treatment for that matter, has traditionally been conducted on a transactional, case-by-case basis. That is, a patient is physically examined by the dentist or other medical specialist, observations are made and duly noted, a diagnosis is made, and a course of treatment is recommended and then followed. The examination is typically a fairly objective process, and is as thorough as the practitioner conducting the examination may care to be. Obviously, such a transactional process will differ between practitioners, each following his or her own level of training and experience. This can result in a practitioner overlooking some physical attributes of the patient and may even result in a patient undergoing a number of different treatments for the same condition, where an alternative diagnosis and treatment in the first instance may have resulted in a better and more expeditious outcome for the patient.

In the view of these inventors, both of whom are experienced and seasoned dental practitioners, what is needed is a method and program for following a comprehensive assessment of a patient's history and physical condition and then generating a diagnostic opinion for that patient. The method and program that is needed would use data input from a variety of sources, including, but not limited to, manual input, radiographic data or by using dental software charting programs, to derive a custom treatment plan for individual patient care. The method and program would also risk assess the treatment derived. To the knowledge of these inventors, there are programs available for gathering data, but not for using that data to formulate a comprehensive treatment plan.

In the experience of these inventors, what is also needed is such a method and system whereby both experienced and inexperienced practitioners can follow a highly structured approach to a patient's treatment. In this way, the practitioner can quickly arrive at a diagnostic opinion without having to implement a number of alternative treatments before arriving at the most beneficial treatment for the patient

Accordingly, it is an object of the present invention to provide a new and useful method and program for collecting data, radiographs, photos, and models, all for the purpose of planning dental treatment for a patient. It is a further object of the present invention to provide such a method and program that assists the dentist in planning dental treatment by charting every aspect of a patient's dental care. It is another object of the present invention to provide such a method and program that will risk assess the dental treatment planned and aid the dentist in restoring the patient's mouth to optimal long term dental health. It is still another object of the present invention to create certain list-handling functions within the program to identify teeth which occur in certain lists but not in others and to identify teeth that have some particular combination of properties. It is yet another object of the present invention to generate an enhanced diagnostic opinion whereby the height of rows that contain merged cells are appropriately formatted and where by dynamic explanatory comments are provided along with the result of particular functions.

### SUMMARY OF THE INVENTION

The present invention has obtained these objects. It provides for a computer implemented method and system whereby dental data for a patient is input via a series of questions and prompts that appear on an active screen through a custom program written in a high level language. The questions answered and the dental data gathered is stored in a relational database, and every answer is given a numerical value. A number of list-handling functions are provided to identify and list teeth in accordance with certain combinations of properties. The values are eventually output to a screen display or spreadsheet where various math functions and custom algorithms are used to populate the display. The output, or report template, generated contains one or more diagnostic opinions that include specific and generalized prognoses and risk assessment for the patient's dental conditions. The database is derived from evidence-based research, which will also be dynamic. The output display is enhanced by use of methods to appropriately format the height of rows that contain merged cells and to provide explanatory comments along with the result of a function.

The foregoing and other features of the method and program of the present invention will become apparent from the detailed description that follows.

### BRIEF DESCRIPTION OF THE DRAWING

FIGS. 1 through 7 are representative pages of the comprehensive assessment worksheet that is used in accordance with the method and program of the present invention to assess the dental condition of a patient by a dentist and to input data relevant to that patient.

FIGS. 8 through 10 are representative screen displays associated with the program that uses the method of the present invention.

FIGS. 11 through 13 are representative pages of the diagnostic opinion output, or report template, that is generated for use by the dentist in accordance with the method and program of the present invention and illustrates the specific and generalized prognosis and risk assessment for the dental patient.

### DETAILED DESCRIPTION

It is to be understood that the method and system of the present invention may be implemented in hardware or software, preferably in computer programs executing on a programmable computer having a processor, a data storage system, at least one input device and at least one output device. Program code is applied to input data to perform the functions described herein and to generate output information. The output information is applied to one or more output devices, in known fashion. The program is preferably implemented in a high level procedural or object oriented programming language to communicate with a computer system. These inventors prefer Visual Basic® (a registered trademark of Microsoft Corporation), a product made by Microsoft that allows the common person to easily make full featured and powerful Windows® (another registered trademark of Microsoft Corporation) programs. However, the program may be implemented in assembly language, machine language, C++ or Java, if desired. In any case, the language may be a compiled or interpreted language. The program is preferably stored on a storage media or device (e.g. ROM or magnetic diskette) readable by a general or special purpose programmable computer, for configuring and operating the computer when the storage media or device is read by the computer to perform the procedures described herein. The inventive system may also be considered to be implemented as a computer-readable storage medium, configured with a computer program, where the storage medium so configured causes a computer to operate in a specific and predefined manner to perform the functions described herein.

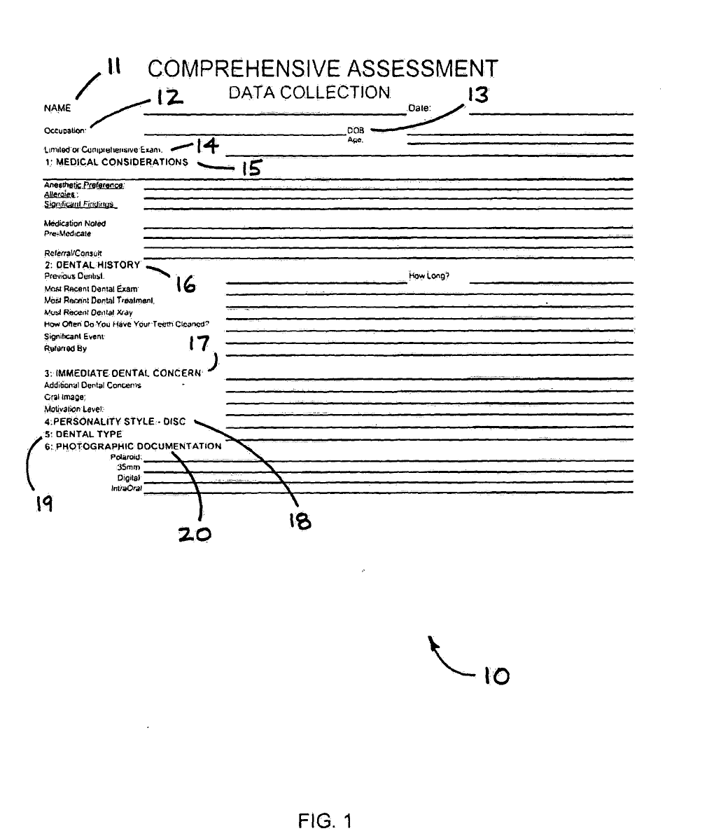

With reference now to the drawings, wherein like numbers represent like elements throughout, FIG. 1 shows the first page of a comprehensive assessment worksheet used in the preferred embodiment of the method and program of the present invention, such first page being identified generally 10. As shown, the first input template page 10 prompts the practitioner for a number of input items, starting with the most basic patient information such as the patient's name 11, his or her occupation 12, his or her date of birth 13 whether or not the examination is “limited” or “comprehensive” 14. The first input template page 10 includes a number of other categories of inquiry entitled “Medical Considerations” 15, “Dental History” 16, “Immediate Dental Concern” 17, “Personality Type” 18, “Dental Type” 19 and “Photographic Documentation” 20. It should be noted that each response to a question or a prompt is given a number in the program and is weighted for importance. The exact number and weight given is not important for purposes of this disclosure because a totally different number and a slightly different weight may be given for different responses depending upon the software instructions given.

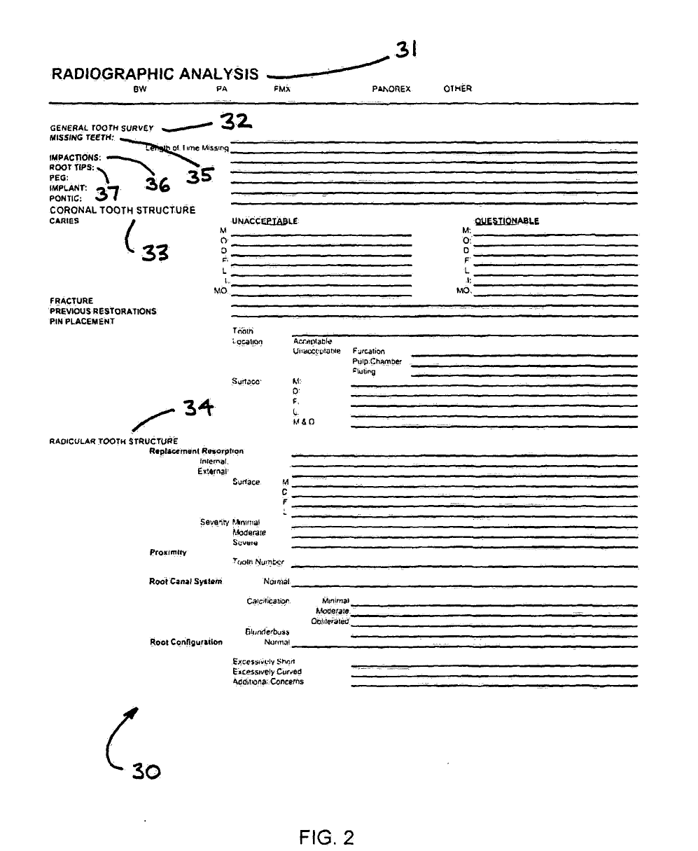

As shown in FIG. 2, a second page of an input template used in the preferred embodiment of the method and system of the present invention is illustrated, such second page being identified generally 30. As shown, the second input template page 30 prompts the practitioner for a number of additional input items, this time specifically directed to “Radiographic Analysis” 31. This input template page 30 also includes prompts for such categories as “General Tooth Survey” 32, “Coronal Tooth Structure” 33, and “Radicular tooth structure” 34. Again, the category “General Tooth Survey” 32 is broken down into a number of subcategories and prompts including “Missing teeth” 35, “Impactions” 36 and “Root tips” 37, among others. Under the subcategory of “Missing teeth” 35, the dentist can identify which teeth are missing according to the number of the tooth and how long it has been missing. It should also be understood that the program and system described can be programmed to identify tooth numbers according to the system that the dentist is accustomed to using. As shown in FIG. 2, this procedure is followed for the remaining radiographic analysis. Again, it should be noted that each response to a question or a prompt is given a number in the program and is weighted for importance. The exact number and weight given as stated above is not important because a totally different number and a slightly different weight may be given for different responses depending upon the software instructions given.

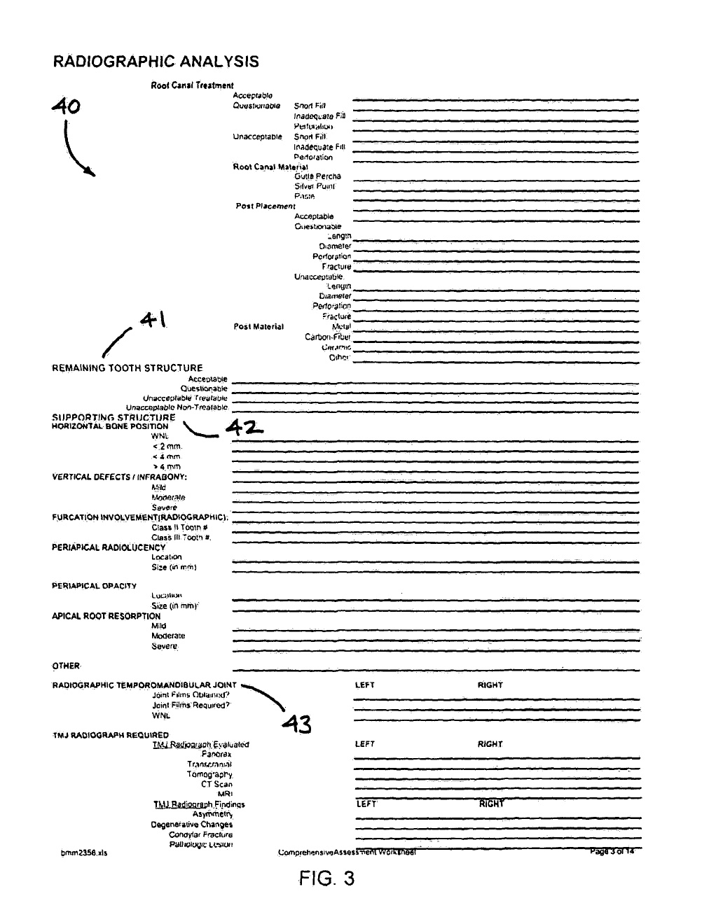

Continuing with FIG. 3, a second page 40 of the radiographic analysis that was initiated with the page 30 as shown in FIG. 2, additional categories of inquiry are made, each with a number of questions and prompts as before. Specifically, the categories include such areas as “Remaining Tooth Structure” 41, “Supporting Structure” 42 and “Radiographic Temporomandibular Joint” 43 and other specific categories and subcategories as shown.

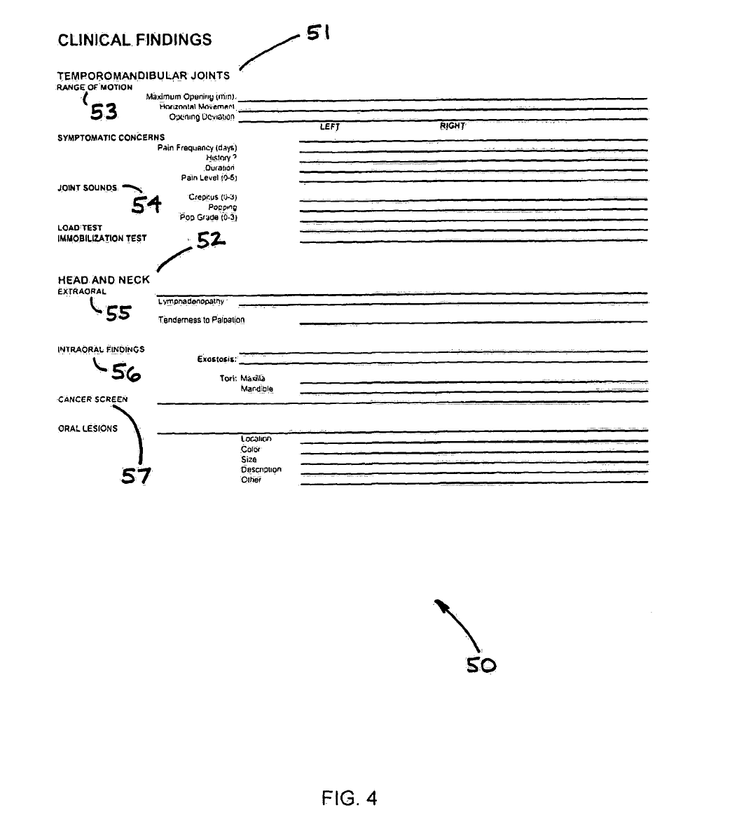

FIG. 4 includes yet another page of questions and prompts for the category “Clinical Findings”, generally identified 50. This page directs the dentist specifically to findings related to the “Temporomandibular Joints” 51 and to the “Head and Neck” 52. Inquiries concerning the temporomandibular joints include “Range of Motion” 53 and “Joint Sounds” 54. Inquiries concerning the head and neck include “Extraoral” 55 and “Intraoral” 56 findings and “Cancer Screen” 57.

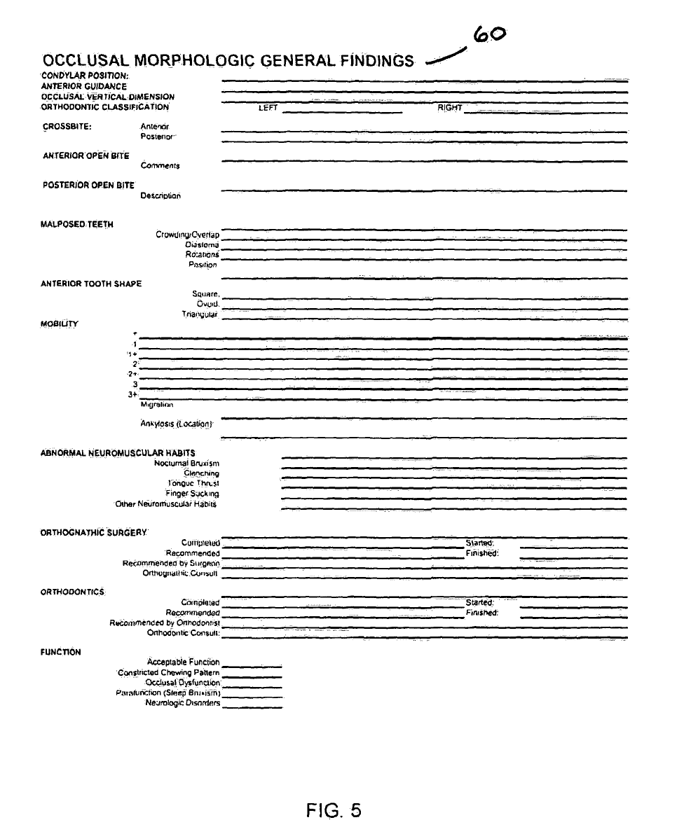

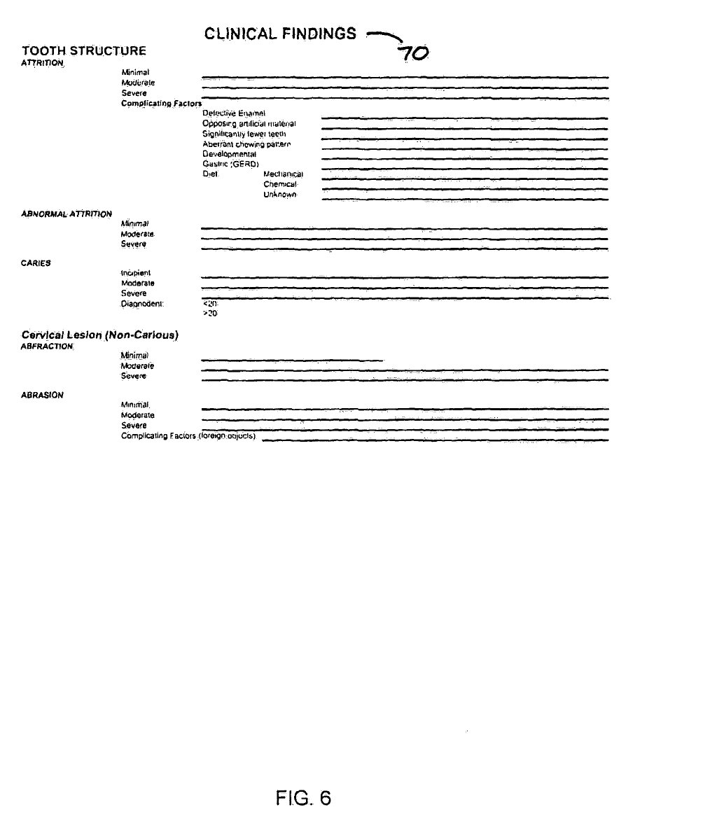

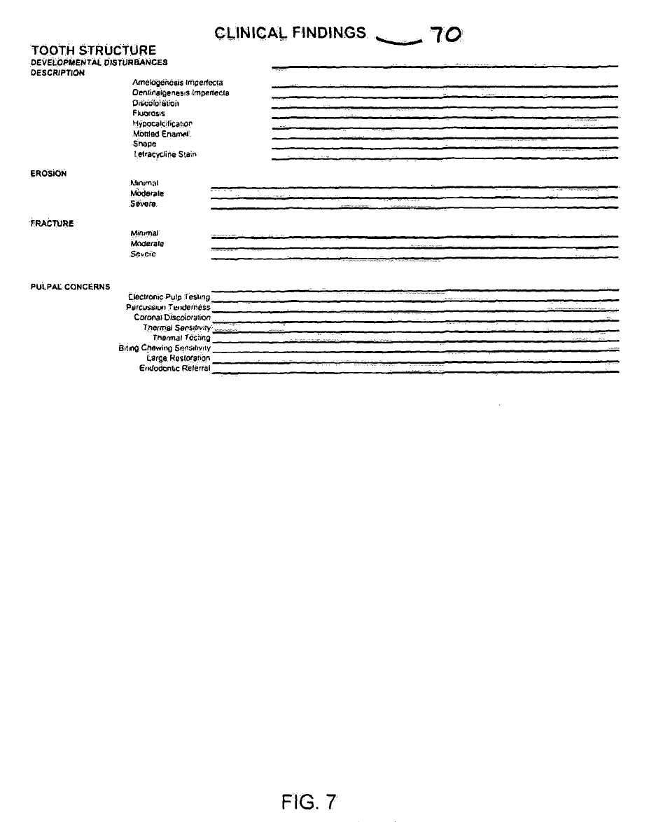

FIG. 5 illustrates another page of questions and prompts in the preferred embodiment of the method and system of the present invention, directed generally to “Occlusal Morphologic General Findings” 60. FIGS. 6 and 7 include prompts and additional questions related to “Clinical Findings”, generally identified 70, and so on. Other pages (not shown) include prompts and questions related to “Direct Restorative Dentistry”, “Indirect Restorative Dentistry”, “Clinical Findings: Periodontal” and “Dentofacial.” Here again, each response to a question or a prompt is given a number in the program and is weighted for importance. The exact number and weight given as stated above is not important because a totally different number and a slightly different weight may be given for different responses depending upon the software instructions given.

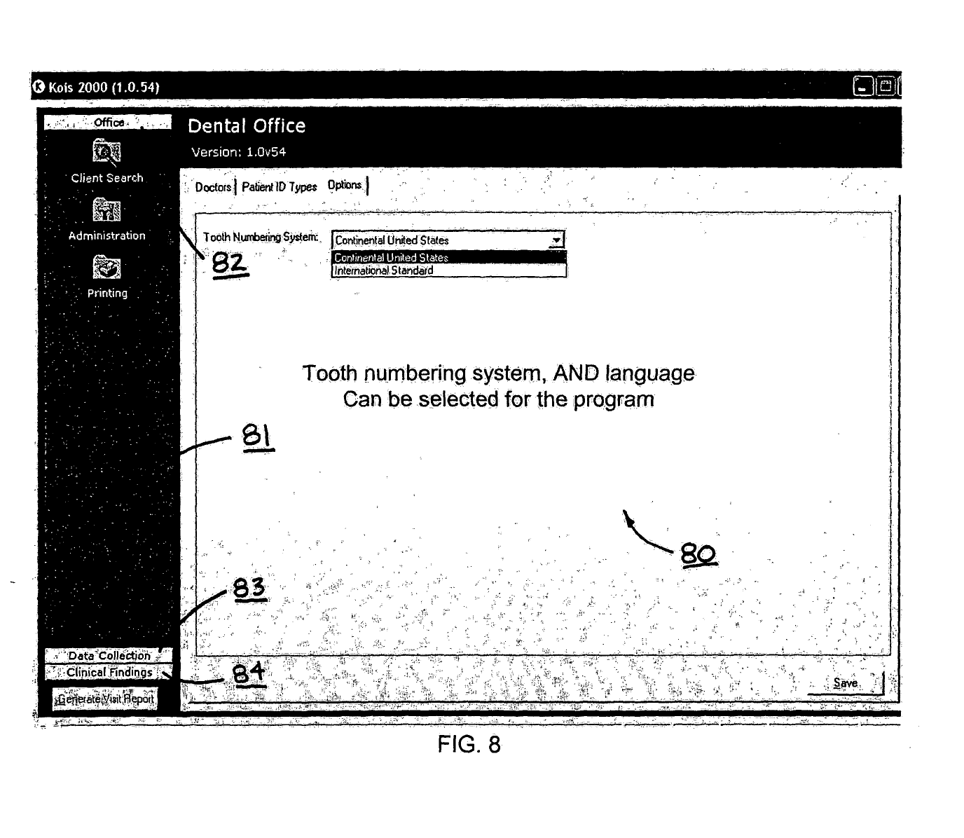

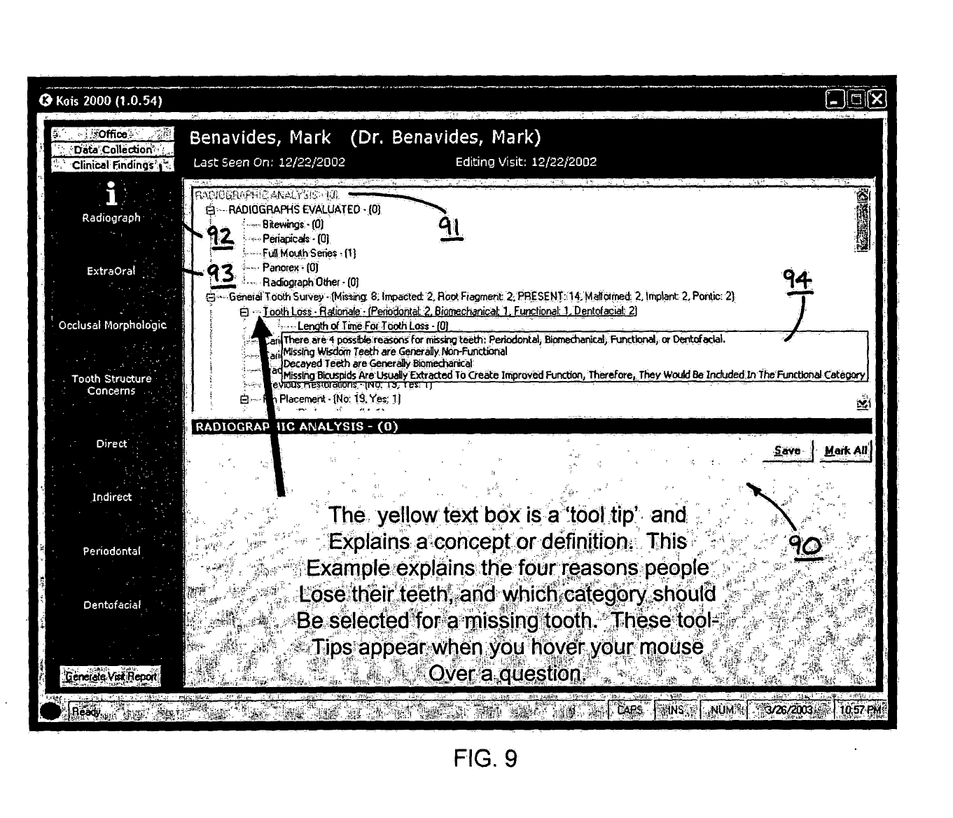

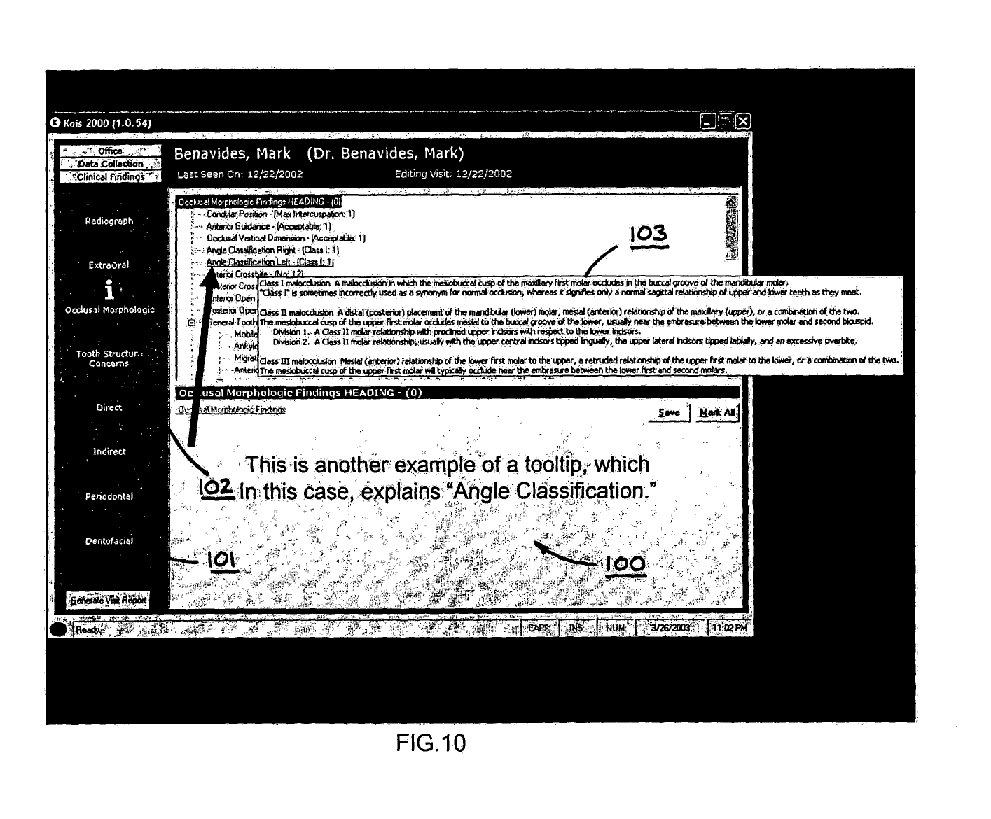

Referring now to FIGS. 8 through 10, representative display screens are shown for the program that is the subject of the present invention. As shown in FIG. 8, the practitioner is provided with a screen display, generally identified 80, having a tool bar 81 from which he or she can select “Office” 82, “Data Collection” 83 or “Clinical Findings” 84. As shown in FIG. 8, this screen display is provided so that a tooth numbering system 80 and a language can be selected by the practitioner using the program. The various assessment worksheets as illustrated in FIGS. 1 through 7 each have screen display counterparts (not shown) that may be accessed while the practitioner is functioning in the “Data Collection” 83 mode. Two other representative displays are shown in FIGS. 9 and 10. FIG. 9 illustrates an active screen display, generally identified 90, of a portion of a “radiographic analysis” output 91 that was the subject of the worksheet that is shown in FIG. 2. The active screen display 90 generally includes information gathered and entered into the system with respect to the particular patient. The “Clinical Findings” tool bar 92 shows what the active screen is and indicates to the user that he or she is in the desired or required area of inquiry, i.e. “Radiograph” 93. As an added feature, the display 90 includes a feature that these inventors refer to as a “tool tip” feature. In the preferred embodiment, the “tool tip” display occurs when the user “hovers” the mouse over an item shown within the active screen display 90. The “tool tip” 94 would appear within the display to explain a concept or definition in dental terms. In the preferred embodiment, the tool tip 94 occurs as a yellow text box although the exact color, format or content of the box is not a limitation of the present invention. FIG. 10 illustrates a portion of the “Clinical Findings” display 100 that is associated with the “Occlusal Morphologic General Findings” sheet 60 shown in FIG. 5. The tool bar 101 shown to one side of the active screen display 100 also tells the user that he or she is working in the “Occlusal Morphologic” 102 area of the program. Another example 103 of the “tool tip” function is shown in FIG. 10 as well.

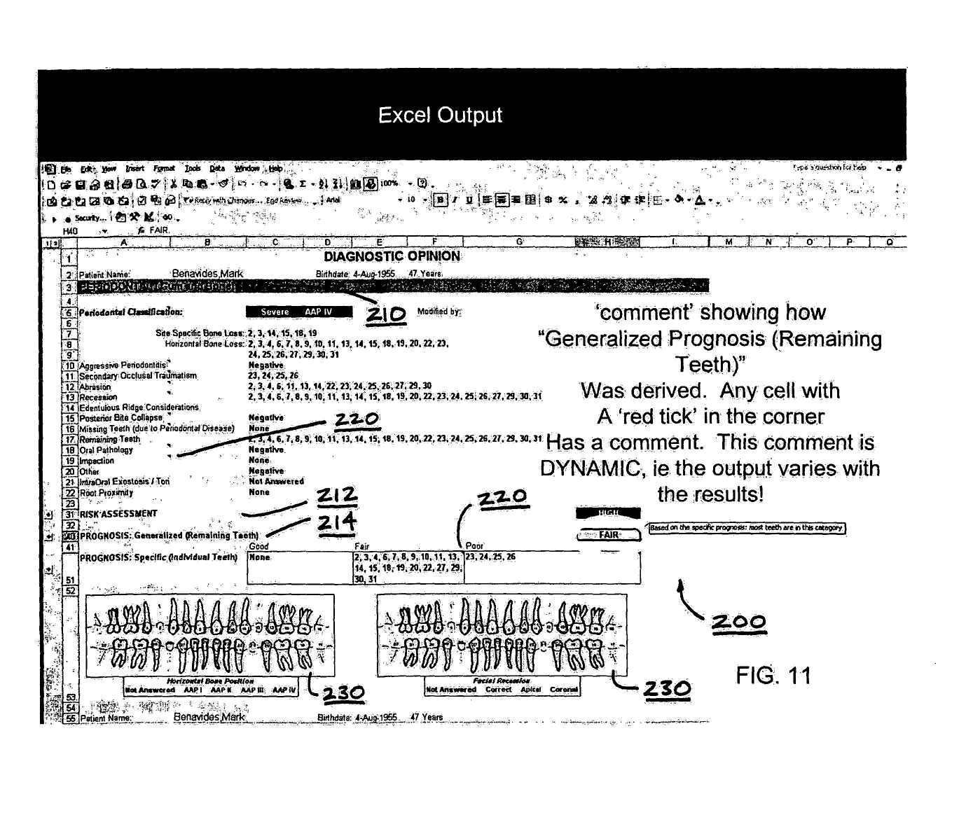

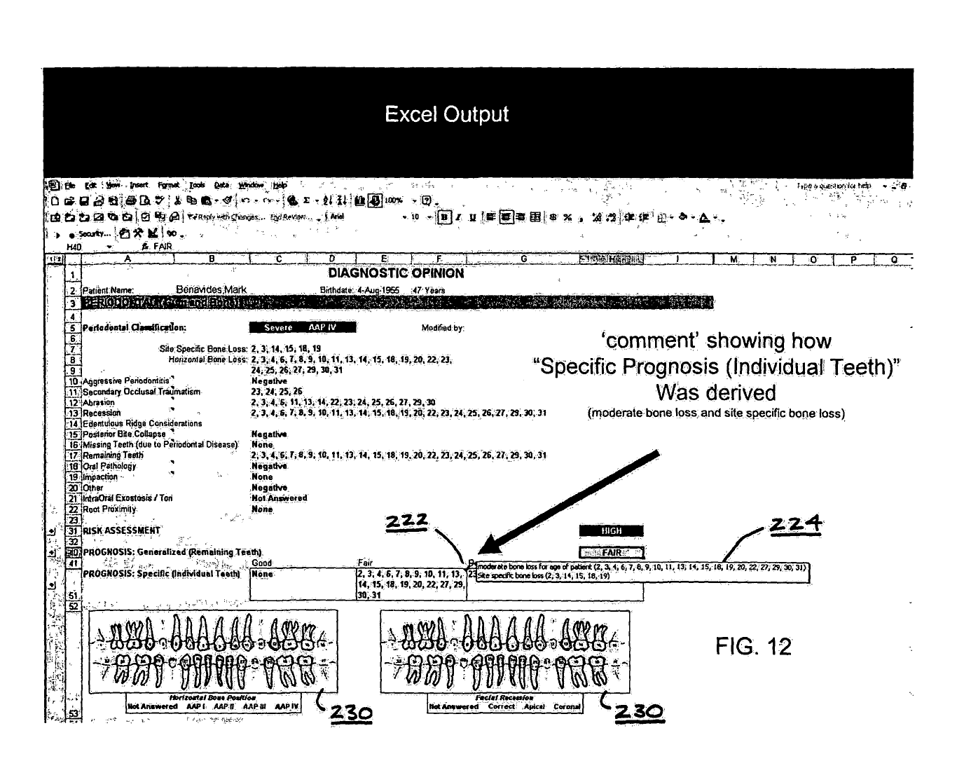

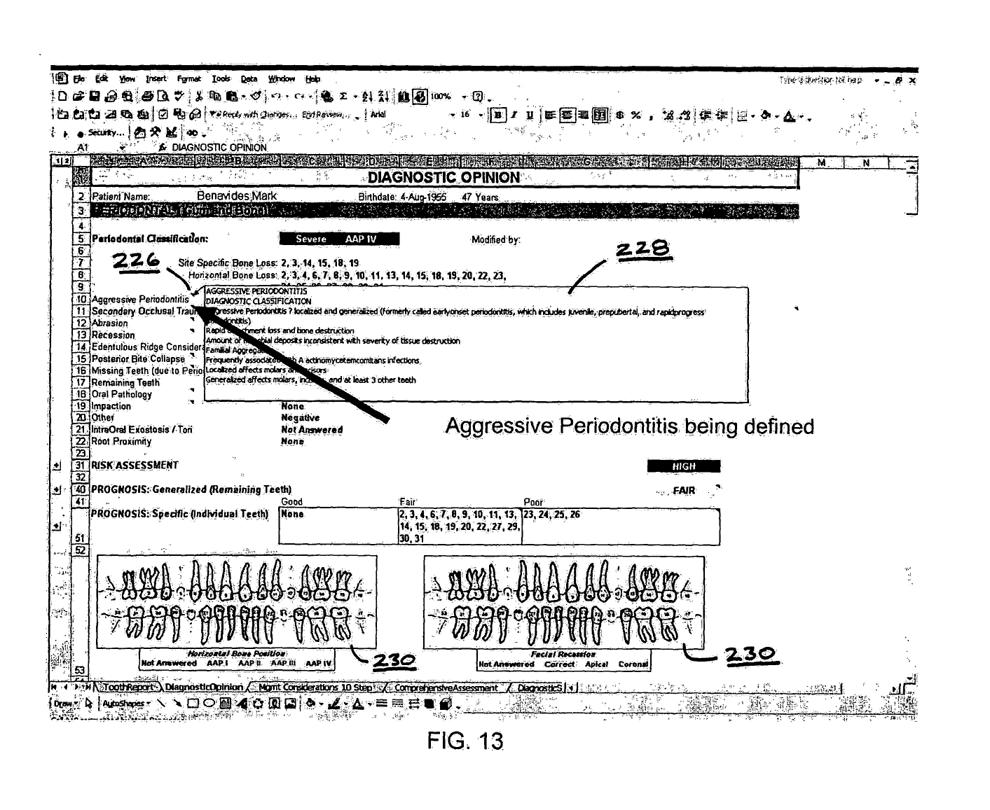

Once the dentist has answered all questions as prompted in accordance with the foregoing scheme of categories and subcategories, the practitioner can generate a “Diagnostic Opinion” display and/or printout. This “Diagnostic Opinion” display, generally identified 200, is dynamic. That is, it changes depending on the input, and is illustrated in the preferred embodiment as a screen of information, shown in FIGS. 11 through 13 to have different capabilities. The page of the display 200, as shown in FIG. 11, addresses “Periodontal (Gum and Bone)” 210 concerns and provides the dentist with a section for “Risk assessment” 212 and “Prognosis” 214. The “Prognosis” and “Risk assessment” sections 212, 214 include a generalized prognosis for all teeth, including those teeth that are missing, and a specific prognosis for individual teeth. In the “Diagnostic Opinion” 200, any cell of information having a red “tick” 220 displayed with the cell indicates that a comment, used to explain results or provide definitions, is also associated with that cell of information. Here again, the color of the “tick” 220 and the nature of the comment is not a limitation of the present invention. However, if no “tick” 220 is shown, then no comment is provided. By way of example, FIG. 12 illustrates one such “tick” 222 and comment 224 associated with the “Prognosis” 214 of certain teeth identified. This comment 224 is in the nature of an explanation of the results of the diagnosis. FIG. 13 illustrates another “tick” 226 and comment 228. That comment 228 is associated with the definition for a term that is contained within the “Diagnostic Opinion” 200. As shown in FIGS. 11 through 13, the “Diagnostic Opinion” 200 also includes visual indicators 230. In the preferred embodiment, the visual indicators 230 are tooth outlines that are color coded to depict additional pathologic tooth structure and other problems that should be addressed by the dentist. The precise color code used is not a limitation of the program and method of the present invention.

One specifically innovative part of the dental software of the present invention is a template for a report. This template is a Microsoft Excel® workbook that contains a number of worksheets which present a Diagnostic Summary, a more detailed Diagnostic Opinion, a Comprehensive Assessment and a set of Management Considerations for a patient, based on data collected during visits by the patient.

The workbook obtains its data from the program database and stores them in various hidden worksheets. In many cases, the data consists of a list of tooth numbers with optional comments.

In creating the “Diagnostic Opinion,” for example, it is necessary to identify teeth which occur in certain lists but not in others, i.e. the teeth which have some particular combination of properties.

The processing of these tooth lists goes beyond the scope of normal Excel formulas. It is carried out by pre-programmed functions written in Visual Basic® for Applications and held within the template workbook. Each result required in the “Diagnostic Opinion” could have had its own specific function, but that would have been expensive to develop and maintain. Therefore a number of general purpose list-handling functions were defined and implemented. These general purpose functions can then be used in normal Excel formulas to produce the desired results.

These inventors are unaware of any similar list-handling functions having been developed previously. The innovation is more in the concept of the set of functions than in the details of their implementation. Other areas of innovation in the report template are in the methods used to appropriately format the height of rows which contain merged cells, and to provide explanatory comments along with the result of a function.

With regard to the tooth list handling functions of the program of the present invention, much of the output is given as lists of teeth. Most of the data imported from the database is also given as lists of teeth.

The format of the imported lists is like:
01 (18),02 (17),03 (16)
Any tooth may have a comment following it.
01 (18)(some comment),02 (19) etc.
A list of no teeth results in an empty cell.
The desired form of the output lists is
1, 2, 3 (for US numbering)
1.8, 1.7, 1.6 (for international numbering)
“None” (where there are no relevant teeth)

Comments are generally not required within the output lists.

“NormaliseList” converts an input list into an output list. It is used whenever an imported list is to be included without further processing in the output sheet.

“NormaliseList” has two arguments:
A string which is usually a single imported list (e.g. in a cell in Main)
An optional default string to be used where there are no teeth in the list. If this parameter is missing, then the default is “None.”

The result is an output list that corresponds directly to the input list, taking account of the current tooth numbering system.

Examples are as follows:
NormaliseList(“01 (18),02 (17),03 (16)”) returns “1, 2, 3” for US numbering or “1.8, 1.7, 1.6” for international numbering
NormaliseList(“”) returns “None”

The remaining examples will use US numbering only.

```
Function NormaliseList(ByVal stList As String, Optional stDefault =
cstNeg) As String
  ′ input lists are “,” separated lists of teeth where each tooth
  ′ is represented as, for example, “01 (18)” where 01 is the US tooth
number
  ′ and 1.8 is the international tooth number
  ′ such a tooth description may be followed by a comment in parentheses
  ′ this procedure turns such a list into a list in output format
  ′ giving tooth numbers in the currently selected numbering system
  ′ removing leading zeros from US tooth numbers
  ′ sorting the tooth numbers into a standard order
  ′ and using “,” as separator
  Dim ST As String
  Dim stNew As String
  Dim i As Integer
  Dim V
  Dim iChar As Integer
  ′ MyReplace is a local equivalent to Replace which is not available in 97
  ′ cstSep is a constant “,” which is the separator used in lists we create
  ′ input lists contain “,” as the separator
  ′ turn all separators back to “,” for now
  ST = MyReplace(stList, cstSep, “,”)
  iChar = InStr(ST, “(”)
  If iChar > 0 Then
    ′ we have some raw data from the export (e.g. “01 (18),02 (17)”
    If ThisWorkbook.Sheets(“Terms”).Range(“Language”) = 0 Then
      ′ US: lose international teeth
      ′ build a replacement string in stNew
      stNew = “”
      Do While iChar > 0
        ′ remove the international tooth numbers “ (18)” etc
        If Mid(ST, iChar + 4, 1) = “)” Then
          stNew = stNew & Left(ST, iChar − 1)
          ST = Mid(ST, iChar + 5)
        Else
          stNew = stNew & Left(ST, iChar + 1) ′ a comment?
          ST = Mid(ST, iChar + 2)
        End If
        iChar = InStr(ST, “(”)
      Loop
      ST = stNew & ST
    Else
      ′ international numbers required
      ′ make an array of the tooth descriptions+comments
      ′ MySplit is a local equivalent to Split which is not available in
      Excel 97
      V = MySplit(ST, “,”)
      ′ look through the array
      For i = LBound(V) To UBound(V)
        ′ if the array element is a tooth number (it might be part of a
        comment)
        ′ then extract the international tooth number (e.g. 18) and add
a central “.”
        If V(i) Like “## (##)*” Then
          V(i) = Mid(V(i), 5, 1) & “.” & Mid(V(i), 6, 1) & Mid(V(i), 8)
        End If
      Next
      ′ sort into standard order
      SortArray V
      stNew = “”
      ′ make into comma separated string of international numbers
      For i = LBound(V) To UBound(V)
        stNew = JoinUp(“,”, stNew, V(i))
      Next
      ST = stNew
    End If
  End If
  ′ put the standard separator back
  ST = MyReplace(ST, “,”, cstSep)
  ′ if there are no teeth in the list then return a default value
  ′ (usually cstNeg which is “None”, but can be specified on the call)
  If ST = “” Then ST = stDefault
  ′ remove leading zeros from teeth names and return the result
  NormaliseList = Mid(MyReplace(cstSep & ST, cstSep & “0”, cstSep),
Len(cstSep) + 1)
End Function
```

“UniqueList” converts a number of lists into a single output list containing each of the teeth that appears in the original lists, removing any duplicates. “UniqueList” requires one or more arguments which can each be either a string or a range of cells which contain either input lists or output lists. The result is an output list.

Examples are as follows:
UniqueList(“1, 2, 3”,“2, 3, 4”) returns “1, 2, 3, 4”
UniqueList(“01 (18),02 (17),03 (16)”, “”, “1, 4”) returns “1, 2, 3, 4”

UniquetList(“”, “None”) returns “None”

```
Function UniqueList(ParamArray P( ))
′ UniqueList is the OR of a set of lists, with each tooth occurring only
once
′ The function can have any number of parameters
′ Each parameter can be a range of cells or a string
  Dim C As Range
  Dim ST As String
  Dim stNew As String
  Dim vVal, vVals
  Dim stLocal As String
  Dim i As Integer
  ′ look at each of the parameters passed
  ′ build in ST a composite list of teeth from all of the parameters
  For i = LBound(P) To UBound(P)
    ′ if it's a range of cells then look at each cell in turn
    If TypeName(P(i)) = “Range” Then
      For Each C In P(i).Cells
        ′ add to ST if there are teeth listed in C
        stLocal = C.Value
        If stLocal <> “” And stLocal <> cstNeg Then
          ST = JoinUp(cstSep, ST, stLocal)
        End If
      Next
    ElseIf TypeName(P(i)) = “String” Then
      ′ a literal string − add to ST if there are teeth listed
      If P(i) <> cstNeg Then ST = JoinUp(cstSep, ST, P(i))
    Else
      ′ if the parameter is not a range or a string it's an error situation
      UniqueList = CVErr(xlErrValue) ′ arg must be a range
      Exit Function ′bm added
    End If
  Next
  ′ if there are no teeth listed in any of the parameters
  ′ return “None”
  If ST = “” Then
    UniqueList = cstNeg
    Exit Function
  End If
  ′ turn the list into normal form
  ST = NormaliseList(ST)
  ′ remove any embedded comments from the list
  ST = StripComments(ST)
  ′ split the list into an array with one tooth per element
  ′ the array may contain duplicates
  vVals = MySplit(ST, cstSep)
  ′ sort teeth into standard order
  SortArray vVals
  ′ rebuild a tooth list in stNew, adding teeth if they are not already listed
  ′ looking at each element of the array
  For Each vVal In vVals
    ′ is the tooth there (surrounded by separators)?
    ′ to simplify the search stNew temporarily has a separator at the
beginning and end
    If InStr(stNew, cstSep & vVal & cstSep) = 0 Then
      ′ if not, add it to stNew maintaining separators at start and end
      If stNew = “” Then stNew = cstSep & vVal & cstSep Else stNew =
stNew & vVal & cstSep
    End If
  Next
  ′ remove the surplus separators from start and end
  stNew = Mid(stNew, Len(cstSep) + 1, Len(stNew) − 2 * Len(cstSep))
  ′ return the result
  UniqueList = stNew
End Function
```

“CountList” counts the number of teeth in a list or a set of lists. “CountList” requires one or more arguments which can each be either a string or a range of cells which contain either input lists or output lists. The result is an integer value which gives the number of teeth in the lists. Note that if a tooth is duplicated on two lists it will be counted twice. The user will use CountList(UniqueList( . . . )) if he or she wants duplicates to be counted only once.

Examples are as follows:
CountList(“01 (18),02 (17),03 (16)”) or CountList(“1, 2, 3”) returns 3
CountList(“”) or CountList(“None”) returns 0
CountList(“1, 2, 3”, “2, 3, 4”) returns 6

CountList(UniqueList(“1, 2, 3”, “2, 3, 4”)) returns 4

```
Function CountList(ParamArray P( )) As Integer
′ add the number of teeth on one or more lists
′ normally used with a single list, but will add the number of teeth on
multiple lists
′ note that with multiple lists duplicates will be counted twice.
  Dim C As Range
  Dim ST As String
  Dim i As Integer
  Dim iCount As Integer
  iCount = 0
  ′ look at each parameter
  For i = LBound(P) To UBound(P)
    If TypeName(P(i)) = “Range” Then
      ′ if it's a range look at each cell
      For Each C In P(i).Cells
        If Not IsEmpty(C) And Len(C.Value) <> 0 And C.Value <> cstNeg
Then
          ′ there are teeth - normalise the list
          ST = NormaliseList(C.Value)
          ′ strip out any embedded comments
          ST = StripComments(ST)
          ′ add the number of teeth in the list to the number so far
          ′ the number of teeth is the number of separators + 1
          iCount = iCount + (Len(ST) − Len(MyReplace(ST, cstSep, “”))) /
Len(cstSep) + 1
        End If
      Next
    Elself TypeName(P(i)) = “String” Then
      ′ if it's a literal string and not empty or “None”
      If P(i) <> “” And P(i) <> cstNeg Then
        ′ as for a cell, normalise, remove comments and add to count of teeth
        ST = NormaliseList(P(i))
        ST = StripComments(ST)
        iCount = iCount + (Len(ST) − Len(MyReplace(ST, cstSep, “”))) /
Len(cstSep) + 1
      End If
    Else
      ′ inappropriate parameter; return error
      CountList = CVErr(xlErrValue) ′ arg must be a range or a string
      Exit Function
    End If
  Next
  ′ return the count
  CountList = iCount
End Function
```

37 CommonList” gives a list of the teeth which are common to a number of lists. “CommonList” requires two or more arguments which can each be either a string or a range of cells which contain either input lists or output lists. The result is an output list which gives the teeth that appear in all of the argument lists. Where an argument is a range of a number of cells (more than one) it will be treated as meaning the “UniqueList” of that set of cells.

Examples are as follows:
CommonList(“01 (18),02 (17),03 (16)”, “1, 3, 4”) returns “1, 3”
CommonList(“1, 2”, “3, 4”) returns “None” because there are no teeth common to the 2 lists

CommonList(“”, “2, 3, 4”) returns “None” because the first list is empty

```
Function CommonList(ParamArray P( ))
′ returns a list of items which occur in lists in all of the parameters passed
′ it's an AND operating on lists.
  Dim C As Range
  Dim ST As String
  Dim stCommon As String
  Dim vVal, vVals
  Dim i As Integer
  Dim j As Integer
  ′ look at each parameter in turn
  For i = LBound(P) To UBound(P)
    ST = “”
    If TypeName(P(i)) = “Range” Then
      ′ if it's a range, produce a composite list in ST
      For Each C In P(i).Cells
        If Not IsEmpty(C) And C.Value <> cstNeg Then
          If ST <> “” Then ST = ST & cstSep
          ST = ST & C.Value
        End If
      Next
    Elself TypeName(P(i)) = “String” Then
      ′ literal string
      If P(i) <> cstNeg Then
        ST = ST & P(i)
      End If
    Else
      ′ unexpected parameter type - return error
      CommonList = CVErr(xlErrValue)
      Exit Function
    End If
    ′ if this parameter has no teeth then the result must be None
    ′ no need to look at the rest.
    If ST = “” Then
      CommonList = cstNeg
      Exit Function
    End If
    ′ turn list into normal form and remove embedded comments
    ST = NormaliseList(ST)
    ST = StripComments(ST)
    ′ if this is the first parameter set stCommon to the list of unique teeth
    If i = LBound(P) Then
      stCommon = UniqueList(ST) ′ remove duplicates
    Else
      ′ make a new stCommon out of the values which are in stCommon and
ST
      ′ first split stCommon into an array of tooth numbers
      vVals = MySplit(stCommon, cstSep)
      ′ now build a new stCommon containing teeth in vVals which are
also in ST
      stCommon = “”
      ′ to simplify matching put a separator before and after ST
      ST = cstSep & ST & cstSep
      ′ look at each tooth in old common list (now in vVals)
      For j = LBound(vVals) To UBound(vVals)
        If InStr(ST, cstSep & vVals(j) & cstSep) > 0 Then
          ′ in common list and in latest parameter; add to stCommon
          If stCommon <> “” Then stCommon = stCommon & cstSep
          stCommon = stCommon & vVals(j)
        End If
      Next
      ′ if there are no teeth in common to all parameters so far
      ′ return “None”; no need to go on.
      If stCommon = “” Then
        CommonList = cstNeg
        Exit Function ′ no point continuing
      End If
    End If
  Next
  ′ return the final common list
  CommonList = stCommon
End Function
```

“ExcludeList” gives a list of the teeth which are on one list and not on any of a number of other lists. “ExcludeList” requires two or more arguments which can each be either a string or a range of cells which contain either input lists or output lists. Where an argument is a range of a number of cells (more than one) it will be treated as meaning the “UniqueList” of that set of cells.

Examples are as follows:
ExcludeList(“01 (18),02 (17),03 (16)”, “1, 3, 4”) returns “2”
ExcludeList(“1, 2”, “3, 4”) returns “1, 2” because there are no teeth common to the 2 lists

ExcludeList(“”, “2, 3, 4”) returns “None” because the first list is empty

```
Function ExcludeList(ParamArray P( ))
′ exclude from the first list any items on the remaining lists
  Dim C As Range
  Dim ST As String
  Dim stMain As String
  Dim vVal, vVals
  Dim i As Integer
  Dim iChar As Integer
  ′ look at each parameter in turn
  For i = LBound(P) To UBound(P)
    If TypeName(P(i)) = “Range” Then
      ′ if the parameter is a range of cells, add a composite list
      ′ from all of those cells in ST
      For Each C In P(i).Cells
        If Not IsEmpty(C) And C.Value <> cstNeg Then
          If ST <> “” Then ST = ST & cstSep
          ST = ST & C.Value
        End If
      Next
    Elself TypeName(P(i)) = “String” Then
      ′ literal string; append any teeth to the list in ST
      If P(i) <> cstNeg Then
        If ST <> “” Then ST = ST & cstSep
        ST = ST & P(i)
      End If
    Else
      ′ unexpected parameter type - return error
      ExcludeList = CVErr(xlErrValue)
      Exit Function
    End If
    ′ ST is now a list of the teeth in this parameter, with possible duplicates
    ′ normalise its presentation and strip out embedded comments
    ST = NormaliseList(ST)
    ST = StripComments(ST)
    If i = LBound(P) Then
      ′ if this is the first parameter then save the list in stMain
      stMain = ST
      ′ if there are no teeth in the first parameter then we know there will
      ′ be none after any exclusions so can return “None” now.
      If stMain = “” Then
        ExcludeList = cstNeg
        Exit Function
      End If
      ′ reinitialise the list so that ST will become a composite list
      ′ of the second and subsequent parameters
      ST = “”
    End If
  Next
  ′ if the list of teeth to exclude from stMain is empty
  ′ then the result is simply stMain.
  If ST = “” Then
    ExcludeList = stMain
    Exit Function
  End If
  ′ otherwise split the teeth to exclude into an array
  vVals = MySplit(ST, cstSep) ′vVals= #'s, delmited by ’,’
  ′ look through the array
  For i = LBound(vVals) To UBound(vVals)
    ′ if the tooth in this array element is in stMain then remove it
    iChar = InStr(cstSep & stMain & cstSep, cstSep & vVals(i) & cstSep)
    If iChar = 1 Then
      ′ it was at the start of stMain so remove the tooth and following
separator
      stMain = Mid(stMain, 1 + Len(cstSep) + Len(vVals(i)))
    Elself iChar > 0 Then
      ′ it was not at the start of stMain so lose the preceding separator and
the tooth
      stMain = Left(stMain, iChar − Len(cstSep) − 1) & Mid(stMain,
iChar + Len(vVals(i)))
    End If
  Next
  If stMain = “” Then
    ′ if there are no teeth left after exclusions applied, return “None”
    ExcludeList = cstNeg
  Else
    ′ otherwise return what remain after exclusions from stMain
    ExcludeList = stMain
  End If
End Function
```

In application, and as part of developing the biomechanical prognosis for specific teeth, anterior teeth are assigned a “Fair” prognosis if they have “Questionable” tooth structure together with any of Endo, PulpPath or Caries. Range names have been defined for the sets of Excel cells containing the lists of teeth with various properties (e.g. Caries), to aid in readability and maintenance of formulas.

In addition, there are functions that return standard lists of teeth in the current numbering system (which is indicated by a cell named Language). Thus AnteriorTeeth(Language) gives “6,7,8,9,10,11,22,23,24,25,26,27” for Language =0 (US numbering) and “1.3,1.2,1.1,2.1,2.2,2.3,3.3,3.2,3.1,4.1,4.2,4.3” for Language=1 (International numbering).

The list of teeth which have any of Endo, PulpPath or Caries are obtained using UniqueList(Endo,PulpPath,Caries_ALL). Since we are only interested in those teeth from this combined list which are also Anterior and have a Questionable tooth structure, we can use CommonList to get the teeth which are common to all three lists:
=CommonList(RemToothStructQuestionable,AnteriorTeeth(Language),Uni queList(Endo,PulpPath,Caries_ALL))

Microsoft Excel® provides a means of wrapping textual data within a cell so that it occupies more than one row. It also provides a means of merging adjacent cells together so that they act as if they were a single cell, and a means of “auto-fitting” the height of a row to the height needed to show all the text in individual cells. Unfortunately auto-fitting of row height does not work with rows containing wrapped text in merged cells.

The report template in the program of the present invention makes frequent use of merged cells to make good use of the width of the page, and often has lists of teeth which extend to more than one line. To produce a nicely formatted report requires a solution to the problem of fitting row heights to wrapped text in merged cells. This was achieved by writing a macro procedure in Visual Basic® for Applications, named SetRowHeights. The present inventors are unaware of anyone having solved this problem previously.

The algorithm used:
looks at each row in turn
identifies those rows which have cells formatted to wrap text
if the row has no merged cells, normal AutoFit is used and, if the row had originally been hidden it is then hidden once more (since AutoFit makes it visible)
if the row has merged cells then it looks at each cell in the row which contains text that is sufficiently long that it may need to have a row of greater than standard height to display all the text. For each such cell it:
   copies the text to a cell in a hidden worksheet
   formats the cell with the same font.
   sets the width of that cell to match the width of the (merged) cell
   autofits the row height to the cell.
   makes an allowance for subsequent rows if the cell is merged vertically
   determines if the row height required for this cell is greater than that required for previous cells
it takes the maximum of the heights computed for the cells in the row and sets the row height to that value.

leaves those rows which have no cells formatted to wrap text at standard height.

```
Sub SetRowHeights(Sh As Object)
′ sets row heights in sheet Sh.
′ Excel doesn't correctly set row height when merged cells have wrapped
text
  Dim C As Range, rRow As Range
  Dim sHeight As Single
  Dim sBestHeight As Single
  Dim bUpdate As Boolean
  Dim bHid As Boolean
  Dim iHidCol As Integer
  Dim cSizer As Range
  ′ switch off screen updating to speed up the process
  bUpdate = Application.ScreenUpdating
  Application.ScreenUpdating = False
  ′ unprotect the Terms worksheet as we modify a text box on that sheet
  With ThisWorkbook.Sheets(“Terms”)
    If .ProtectContents Then .Protect userinterfaceonly:=True,
password:=cPass
    ′ we will use a cell on this other sheet to get the right height
    Set cSizer = .Range(“RowSizer”)
  End With
  ′ this process is only relevant to worksheets, not chart sheets
  If TypeName(Sh) = “Worksheet” Then
    If IsNull(Sh.UsedRange.WrapText) Then
      ′ text wrapping done in some cells in the sheet so unprotect the sheet
      ′ to allow the code to change the row heights.
      If Sh.ProtectContents Then Sh.Protect userinterfaceonly:=True,
password:=cPass
      ′ look at each row in turn
      For Each rRow In Sh.UsedRange.Rows
        If IsNull(rRow.WrapText) Then
          ′ there are cells on this row with wrapped text
          On Error Resume Next
          ′ note if the row is hidden by an outline
          ′ (because setting row height will make it visible)
          bHid = rRow.SpecialCells(xlVisible).Count = 0
          If Err <> 0 Then bHid = True
          On Error GoTo 0
          If Not IsNull(rRow.MergeCells) Then
            ′ no merged cells so can use Excel's autofit
            rRow.EntireRow.AutoFit
            ′ and hide the row again if necessary now it has correct height
            rRow.EntireRow.Hidden = bHid
          Else
            ′ row has merged cells and wrapped text
            sBestHeight = 12.75
            For Each C In rRow.Cells
              ′ copy the content of the cell to a spare cell in Terms and Autofit
there
              If C.Address = C.MergeArea.Range(“A1”).Address _{—}
                  And C.WrapText And Not C.EntireColumn.Hidden Then
                ′ first of a merged cell, or a single cell, with wrapped text
                ′ and column not hidden
                ′ set the single cell in Terms to match the (merged) cell here
                cSizer.Value = C.Text
                cSizer.Font.Size = C.Font.Size
                cSizer.Font.Bold = C.Font.Bold
                ′ Width is measured in Twips; we can read width of the
MergeArea
                ′ but we can only set the ColumnWidth which is measured in
different units
                ′ so scale the Width appropriately
                cSizer.EntireColumn.ColumnWidth = C.MergeArea.Width *
cSizer.ColumnWidth / cSizer.Width
                cSizer.WrapText = True
                ′ use AutoFit to find the right row height for this cell
                cSizer.EntireRow.AutoFit
                ′ get the height
                sHeight = cSizer.RowHeight
                ′ if the cell is merged vertically then we need less height than
                this
                If C.MergeArea.Rows.Count > 1 Then
                  ′ adjust height down for later rows
                  sHeight = sHeight − (C.MergeArea.Rows.Count − 1) *
(C.Font.Size + 2.75)
                End If
              Else
                sHeight = C.Font.Size + 2.75
              End If
              ′ take the greatest height for this row so far
              If sHeight > sBestHeight Then sBestHeight = sHeight
            Next
            ′ if the row isn't the correct height
            If rRow.EntireRow.RowHeight <> sBestHeight Then
              ′ set it to the correct height
              rRow.EntireRow.RowHeight = sBestHeight
              ′ and hide it again if appropriate
              rRow.EntireRow.Hidden = bHid
            End If
          End If
        End If
        ′ additional test for rows which are conditionally hidden
        ′ if that column on this row contains a True/False value
        If Sh.Name = “DiagnosticOpinion” Or Sh.Name = “ToothReport”
        Then
          ′ there is a column containing a cell named “Hidden”
          ′ get its column number
          If iHidCol = 0 Then iHidCol = Sh.Range(“Hidden”).Column
          If TypeName(rRow.Cells(1, iHidCol).Value) = “Boolean” Then
            ′ if the cell on this row in the Hidden column is True/False
            ′ then hide the row (True) or reveal it (False)
            rRow.EntireRow.Hidden = rRow.Cells(1, iHidCol).Value
          End If
        End If
      Next
    End If
  End If
  ′ restore screenupdating to its previous state
  Application.ScreenUpdating = bUpdate
End Sub
```

The “Diagnostic Opinion” sheet in the report template gives a number of prognoses and risk assessments. It was desired to show the reasoning that led to those results, linked in some way to the result. Use of adjacent cells would have resulted in an unattractive layout. The preferred method was to use Excel® cell comments, which pop up when the mouse cursor points to the cell concerned.

It is generally believed that Microsoft Excel® user-defined functions written in Visual Basic® for Applications cannot make changes to the Excel® environment other than returning a value into the cell from which the function was called. In particular, such a function cannot change the formatting of a cell, or change the value of any other cell, or perform an action such as selecting a different worksheet. It was discovered that it is possible for a user-defined function to create/modify a cell comment in the cell containing the call of the function. This technique was used in a number of functions, “RiskReport”, “PrognosisReport”, “SpecificReport” and “AddAComment.”

“RiskReport” takes two arguments, a range of cells containing risk assessments for each of a number of rows, and a value representing the highest risk assessed on any of the rows. For each cell in the range, if the risk assessment matches the highest assessment, a suitable explanatory text string is accessed from an adjacent column on the same row and appended to any previous such strings. The resultant string is added to the cell containing the function call as a cell comment. The function result is the highest risk.

```
Function RiskReport(ByVal Risks As Range, ByVal Worst As Integer)
′ Risks is a range of cells containing risk designations in column 1
′ and descriptions of the causes of the risk assessments in column 4
′ Worst is the index in the Risk table of the highest risk from that range
′ (computed in the worksheet)
  Dim C As Range
  Dim ST As String
  Dim stWorst As String
  If Worst = 0 Then
    ′ if no risks were identified then Worst will be 0
    ′ treat as Low risk
    stWorst = UCase(ThisWorkbook.Sheets(“Terms”).Range(“t_Low”))
  Else
    ′ otherwise use Worst to select the description
    ′ of the highest level of risk encountered
    ′ from the Risk table
    stWorst = Range(“Risk”).Cells(Worst, 1).Value
  End If
  ′ now look at each cell in column 1 of Risks in turn
  ′ (they contain risk descriptions)
  For Each C In Risks.Columns(1).Cells
    ′ for each one which matches the highest risk, take the descriptive text
from
    ′ 3 columns to the right and add it to a string which will form the
    comment
    If C.Value = stWorst Then
      ′ insert a newline between each pair of strings.
      ST = JoinUp(Chr(10), ST, C.Offset(, 3))
    End If
  Next
  ′ add the result as a comment on the cell
  AddAComment ST
  ′ return the text for the worst risk into the cell.
  RiskReport = stWorst
End Function
“PrognosisReport” works similarly to “RiskReport” for general prognoses.
Function PrognosisReport(ByVal Prognoses As Range, ByVal Worst As
Integer)
′ Prognoses is a range of cells containing prognosis designations in
column 1
′ and descriptions of the causes of the prognoses in column 4
′ Worst is the index in the Prognosis table of the worst prognosis
′ from that range
′ (computed in the worksheet)
  Dim C As Range
  Dim ST As String
  Dim stWorst As String
  If Worst = 0 Then
    ′ if no prognoses were identified then Worst will be 0
    ′ treat as Good prognosis
    stWorst = UCase(ThisWorkbook.Sheets(“Terms”).Range(“t_Good”))
  Else
    ′ otherwise use Worst to select the description
    ′ of the worst prognosis encountered
    ′ from the Prognosis table
    stWorst = Range(“Prognosis”).Cells(Worst, 1).Value
  End If
  ′ now look at each cell in column 1 of Prognoses in turn
  ′ (they contain prognosis designations like FAIR)
  For Each C In Prognoses.Columns(1).Cells
    ′ for each one which matches the worst prognosis,
    ′ take the descriptive text from
    ′ 3 columns to the right and add it to a string which will form the
    comment
    If C.Value = stWorst Then
      ′ insert a newline between each pair of strings.
      ST = JoinUp(Chr(10), ST, C.Offset(, 3))
    End If
  Next
  ′ add the result as a comment on the cell
  AddAComment ST
  ′ return the text for the worst prognosis into the cell.
  PrognosisReport = stWorst
End Function
```

“SpecificReport” is used for the tooth-specific prognoses. It has three arguments: a tooth list to be used as the result of the function—showing the teeth with a particular prognosis (Good, Fair, Poor, Hopeless), a range of cells containing tooth lists for that prognosis, and a range of cells containing suitable explanatory texts for each of the individual tooth lists. In the specific prognosis, each tooth will appear in the worst prognosis category that applies to it, and it may have a number of reasons for being in that category. The cell comment is constructed by using “CommonList” to identify teeth which are in the result list and also in a specific cell from the range of tooth lists for that prognosis; if there are any, the list of such teeth is appended together with the explanatory text to the cell comment.

```
Function SpecificReport(ToothList As String, Contributors As Range,
Reasons As Range)
′ used for the summary line in specific prognosis tables
′ ToothList is the computed tooth list for this prognosis
′ - using ExcludeList to exclude teeth which have worse specific
prognoses.
′ Contributors is a range of cells containing tooth lists
′ with the relevant prognosis
′ Reasons is a corresponding range of cells containing
′ the reasons for those prognoses
  Dim C As Range
  Dim ST As String
  Dim stList As String
  Dim iCell As Integer
  ′ look at each cell in the Contributors range
  For iCell = 1 To Contributors.Cells.Count
    If Not IsEmpty(Reasons(iCell)) Then
      ′ if there are teeth in common between the list in that cell and the
result list
      stList = CommonList(ToothList, Contributors(iCell))
      If stList <> cstNeg Then
        ′ there are some here
        ′ add the corresponding reason to the comment
        ′ (followed by tooth list in brackets)
        ′ and separate different prognoses with a newline.
        ST = JoinUp(Chr(10), ST, Reasons(iCell) & “ (“ & stList & ”)”)
      End If
    End If
  Next
  ′ add the resulting comment to the cell
  AddAComment ST
  ′ return the result list to the cell
  SpecificReport = Tooth List
End Function
```

Each of the earlier functions uses “AddAComment” to actually set up the comment in the cell. “AddAComment” can also be called directly from a worksheet formula. It returns an empty string (“”) as a result but sets the comment on the cell to whatever is passed to it as an argument. For example:

=UniqueList(A, B)&AddAComment(“These teeth satisfy conditions A and B”)

```
Function AddAComment(ByVal ST As String)
′ can be called from within a worksheet formula
′ or from a UDF that has been called from a worksheet formula
  With Application.Caller
      ′ Application.Caller is the cell containing the call of the function
    If Not .Parent.ProtectContents Then
      ′ the Parent is the worksheet.
      ′ Once it is protected the report is frozen
      ′ and the comment need not be changed.
      If Not .Comment Is Nothing Then
        ′ if the cell already has a comment, see if it is what we want it to be
        ′ if so, no more to do
        If ST = .Comment.Text Then Exit Function
        ′ otherwise clear the existing comment from the cell
        .ClearComments
      End If
      ′ if a comment is required on the cell, add it.
      If ST <> “” Then
        .AddComment ST
      End If
    End If
  End With
  ′ return null string so that AddAComment(“xxx”)
  ′ can be appended to any formula
  AddAComment = “”
End Function
```

From the foregoing detailed description of the illustrative embodiment of the invention set forth herein, it will be apparent that there has been provided a new and useful method and program for collecting data, radiographs, photos, and models, all for the purpose of planning dental treatment for a patient; that assists the dentist in planning dental treatment by charting every aspect of a patient's dental care; that will risk assess the dental treatment planned and aid the dentist in restoring the patient's mouth to optimal long term dental health

1. A computer implemented method for generating a diagnostic opinion for treatment of a dental patient, comprising the steps of:
providing a detailed series of questions to be asked of a patient,
providing a computer having at least one assessment function based on gathered physical condition and history information for a patient,
inputting responses from the patient to such questions into the computer,
generating in the computer a diagnostic opinion for the patient.

2. The method of claim 1 wherein said diagnostic opinion generating step includes providing both specific and generalized prognoses relative to the patient as a result of the at least one assessment function.

3. The method of claim 2 wherein said diagnostic opinion generating step includes risk assessing one or more of the patient's conditions.

4. The method of claim 3 wherein the computer providing step includes providing at least one list-handling function whereby specific teeth may be identified as occurring in certain lists and be combined with others having like properties.

5. The method of claim 4 wherein the diagnostic opinion generating step includes providing one or more explanatory comments along with the opinion.

6. The method of claim 5 wherein the diagnostic opinion generating step includes the generation of a report template whereby the height of rows that contain textual data within merged cells are autofitted.

7. The method of claim 6 wherein said question providing step includes providing a plurality of questions and prompts concerning one or more of the areas of
(a) dental history,
(b) dental concern,
(c) radiographic analysis,
(d) supporting structure,
(e) radiographic temporomandibular joint,
(f) clinical findings of the head and neck,
(g) clinical findings of the temporomandibular joints,
(h) occlusal morphologic general findings,
(i) clinical findings as to tooth structure,
(j) direct restorative,
(k) indirect restorative,
(l) clinical findings for periodontal concerns, and
(m) dentofacial concerns.

8. A program for use with a computer in providing dental care comprising
a user interface for enabling the input of information relative to a dental patient,
a database for assigning points to certain answers and for weighting certain answers,
means for generating a diagnostic opinion for the dental patient.

9. The computer program of claim 8 wherein said opinion generating means includes various math functions and custom algorithms to populate at least one worksheet.

10. The computer program of claim 9 wherein said opinion generating means further includes means for generating one or more diagnostic opinions that include specific and generalized prognoses for the patient's conditions.

11. The computer program of claim 10 wherein said opinion generating means further includes means for risk assessing one or more of the patient's conditions.

12. The computer program of claim 11 wherein the opinion generating means includes at least on list-handling function where by specific teeth may be identified as occurring in certain lists and be combined with other having like properties.

13. The computer program of claim 12 wherein the opinion generating means further includes means for providing one or more explanatory comments along with the opinion.

14. The computer program of claim 13 wherein the opinion generating means further includes means for generating a report template whereby the height of rows that contain textual data within merged cells are autofitted.

15. The computer program of claim 14 wherein the user interface includes a series of questions and prompts to be asked of a patient.

16. The computer program of claim 15 wherein the questions and prompts concern one or more of the areas of
(a) dental history,
(b) dental concern,
(c) radiographic analysis,
(d) supporting structure,
(e) radiographic temporomandibular joint,
(f) clinical findings of the head and neck,
(g) clinical findings of the temporomandibular joints,
(h) occlusal morphologic general findings,
(i) clinical findings as to tooth structure,
(j) direct restorative,
(k) indirect restorative,
(l) clinical findings for periodontal concerns, and
(m) dentofacial concerns.

17. A computer program, such program residing on a computer-readable medium, for health management, comprising instructions for causing a computer to
prompt the acceptance of health information relative to a patient by a health care provider,
accept health history, medical information and other considerations relevant to treatment of the patient concerned,
utilize at least one algorithm based on the accepted patient information for generating a diagnostic opinion for that patient.

18. The computer program of claim 17 wherein said algorithm utilizing step includes risk assessing the patient's condition.

19. The computer program of claim 18 wherein the application is to a dental patient by a dentist or a dental practitioner.

20. The computer program of claim 19 wherein the health information inputted concerns one or more of the areas of
(a) dental history,
(b) dental concern,
(c) radiographic analysis,
(d) supporting structure,
(e) radiographic temporomandibular joint,
(f) clinical findings of the head and neck,
(g) clinical findings of the temporomandibular joints,
(h) occlusal morphologic general findings,
(i) clinical findings as to tooth structure,
(l) direct restorative,
(k) indirect restorative,
(l) clinical findings for periodontal concerns, and
(m) dentofacial concerns.

21. The computer program of claim 20 wherein the algorithm utilizing step includes at least one list-handling function whereby specific teeth may be identify as occurring in certain lists and be combined with others having like properties.

22. The computer program of claim 21 wherein the diagnostic opinion that is generated includes explanatory comments along with the opinion.

23. The computer program of claim 22 wherein the diagnostic opinion includes a report template whereby the height of rows that contain textual data within merged cells are autofitted.
